(19) United States
(12) Patent Application Publication
(10) Pub. No.: US 2016/0257027 A1
(43) Pub. Date: Sep. 8, 2016

(54) SCREW EXTRUDER

(71) Applicants: SHOWA DENKO K.K., Manato-ku, Tokyo (JP); THE JAPAN STEEL WORKS, LTD., Shinagawa-ku, Tokyo (JP)

(72) Inventors: Ichiro FUJITA, Tokyo (JP); Shinji YAMAKI, Tokyo (JP); Tadashi YONEDA, Tokyo (JP); Daigo SAGA, Hiroshima-shi (JP)

(73) Assignees: SHOWA DENKO K.K., Tokyo (JP); THE JAPAN STEEL WORKS, LTD., Tokyo (JP)

(21) Appl. No.: 15/027,640

(22) PCT Filed: Sep. 9, 2014
(86) PCT No.: PCT/JP2014/073793

§ 371 Date: Apr. 6, 2016

(30) Foreign Application Priority Data
Oct. 7, 2013 (JP) 2013-210209

(51) Int. Cl. B29B 7/48; C12P 19/02; B01J 16/00; C12P 19/14; B29B 7/60; B29B 7/82

(52) U.S. Cl. CPC B29B 7/482; B29B 7/60; B29B 7/826; B29B 7/488; B01J 16/00; C12P 19/14; C12P 19/02; C12P 2201/00

(57) ABSTRACT

A screw extruder for industrially mass-producing a cellulose-containing composition having a high saccharification performance by continuously conducting pretreatment of cellulose-containing biomass to pretreatment, which screw extruder is characterized as including a raw-material feed portion, a pulverization section, a heating section and a cooling section, and having a plurality of seal rings arranged in the heating section.

### TECHNICAL FIELD

The present invention relates to screw extruder for continuously subjecting a cellulose-containing biomass to hydrothermal treatment to thereby produce a biomass composition having high saccharification performance from a cellulose-containing biomass to glucose.

### BACKGROUND ART

As part of measures against global warming, there have been wide investigations on production of various chemical products including ethanol and the like through effective utilization of cellulose-containing biomass. Examples of the cellulose-containing biomass include hard biomass such as cedar or cypress, and soft biomass, such as rice straw, wheat straw, corncobs, cassava, bagasse, or sugar cane leaves. The biomass may contain hemicellulose, lignin, and the like, and hence it is difficult to directly saccharify the biomass. Therefore, there have been proposals to enhance its saccharification performance through various pretreatments.

As a pretreatment method for enhancing the saccharification performance, there have been proposed a method involving adding an acid or alkali and performing hydrothermal treatment, and a method involving a combination of hydrothermal treatment without a chemical and physical pulverization treatment (JP 2006-136263 A; Patent Document 1). Further, in addition to those methods, there have been proposed water vapor blasting, ammonia blasting, ozone oxidation, white-rot fungus treatment, microwave irradiation, electron beam irradiation, and y-ray irradiation (Journal of The Japan Wood Research Society, 53, 1-13 (2007); Non-Patent Document 1).

However, when those pretreatment methods are studied for industrially useful treatment steps, a device and a method capable of continuously and efficiently treating a raw material in a large amount are not specifically disclosed, while those methods each have an effect of enhancing the saccharification performance to some extent.

As a method of continuously and efficiently performing pretreatment prior to enzymatic saccharification of biomass, in JP 59-192093 A (Patent Document 2), and JP 59-192094 A and JP 2012-170355 A (Patent Documents 3 and 4; U.S. Pat. No. 4,642,287), it has been proposed that a pretreatment method involving kneading biomass with an alkali and subjecting the biomass to hydrothermal treatment with a twin-screw extruder can be continuously performed at a high concentration in a short treatment time as compared to conventional fine pulverization treatment and alkali steam treatment.

However, the pretreatment methods disclosed in Patent Documents 2 to 4 each involve high chemical cost owing to the use of the alkali at a ratio of around 20% with respect to a raw material, and inevitably require neutralization and washing of the added alkali prior to the enzymatic saccharification. Therefore, a problem in terms of economy and efficiency including even a saccharification step is not solved. Further, the pretreatment methods of Patent Documents 2 and 3 are each disclosed as substantially a combination of pulverization and alkali steaming, but with regard to the treatment conditions, only the conditions related to the alkali steaming, such as a heating temperature, a heating time, and the amounts of the raw material and the alkali to be loaded, are presented, and the configuration of a device related to the pulverization, etc. are not presented. The embodiments for carrying out those inventions are unclear.

As a method of easily and rapidly performing pretreatment of plant biomass, in JP 2011-130745 A (Patent Document 5), there has been proposed a method involving sequentially performing pretreatment operations in an extruder in a continuous manner, the operations involving adding a decomposer to plant biomass coarsely pulverized into a preset size or less and subjecting the plant biomass to pressurized hot water treatment, and subsequent operations before saccharification loading in which the plant biomass is mixed with an enzyme for saccharification. However, in Patent Document 5, the conditions under which the treatment method is performed, and data on saccharification of the biomass as to what level of saccharification performance is obtained are not presented, while a flowchart and a screw configuration of the extruder for the method are disclosed. The overall performance and efficiency including even a saccharification step are unclear.

In view of the foregoing, there is a demand for establishment of an industrially useful device for conducting pretreatment for cellulose-containing biomass, which is capable of continuously treating a raw material in a large amount, and enables high saccharification performance of a treated product and high overall production efficiency including even sugar production.

### PRIOR ART

### Patent Document

Patent Document 1: JP 2006-136263 A
Patent Document 2: JP 59-192093 A (U.S. Pat. No. 4,642,287)
Patent Document 3: JP 59-192094 A (U.S. Pat. No. 4,642,287)
Patent Document 4: JP 2012-170355 A
Patent Document 5: JP 2011-130745 A
Non-Patent Document
Non-Patent Document 1: Journal of the Japan Wood Research Society, 53, 1-13 (2007)

### DISCLOSURE OF INVENTION

### Problem to be Solved by Invention

An object of the present invention is to provide an industrially useful treatment device which is capable of continuously conducting pretreatment of a cellulose-containing biomass in large quantity for obtaining a cellulose-containing composition having a high saccharification performance.

### Means to Solve Problem

The inventors of the present invention have made extensive investigations in order to achieve the above-mentioned object. As a result, the inventors have found that, a screw extruder provided with a plurality of seal rings in a heating section is effective as a pretreatment device for obtaining a sugar from a cellulose-containing biomass, and have accomplished the present invention.

That is, the present invention relates to the following pretreatment device for cellulose-containing biomass.
[1] A screw extruder, which is a screw extruder for continuously conducting hydrothermal treatment of cellulose-containing biomass to produce a biomass composition for saccharification, and comprises a raw-material feeding section, a pulverization section, a heating section and a cooling section, characterized in that a plurality of seal rings are provided in the heating section.
[2] The screw extruder as described in [1] above, wherein three sets or more elements each comprising a seal ring and at least one set of a kneading disc and/or a left-hand screw arranged immediately upstream of the seal ring are arranged in the heating section.
[3] The screw extruder as described in [2] above, wherein the clearance between the seal ring arranged in the heat section and a cylinder is 0.5% to 10.0% with respect to the inner diameter of the cylinder.
[4] The screw extruder as described in any one of [1] to [3] above, wherein at least one or more elements each comprising a seal ring and at least one set of a kneading disc and/or a left-hand screw is arranged in the pulverization section.
[5] The screw extruder as described in any one of [1] to [4] above, wherein a liquid feed line is arranged in the pulverization section.
[6] The screw extruder as described in any one of [1] to [5] above, wherein a water cooling jacket and/or a liquid feed line are arranged in the cooling section.
[7] The screw extruder as described in any one of [1] to [6] above, wherein a compactor is provided in the raw-material feeding section.
[8] The screw extruder as described in any one of [1] to [7] above, wherein an L/D as being an effective length represented by a ratio between the length (L) of a screw, which is measured from a start point of screw thread below the hopper to the tip of the screw, and the diameter (D) of the screw, is from 30 to 80 in its entirety including the pulverization section, the heating section and the cooling section, and from 10 to 40 in the pulverization section.
[9] The screw extruder as described in any one of [1] to [8] above, wherein the screw extruder is a co-rotating twin-screw extruder.
[10] The screw extruder as described in any one of [1] to [9] above, wherein the cellulose-containing biomass is soft biomass.

### Effects of Invention

The screw extruder of the invention, in which a plurality of seal rings are arranged in the heating section, makes it possible to obtain a cellulose-containing composition having a high saccharification performance by continuously conducting pretreatment of cellulose-containing biomass to pretreatment in large quantity, and enables establishment of an industrially useful process for producing a sugar, which is high not only in saccharification performance of a treated product but also in overall production efficiency including even sugar production.

### BRIEF DESCRIPTION OF DRAWINGS

FIG. 1(A) is a configuration view of a screw extruder A used in Examples 2, 4 and 5, FIG. 1(B) is a configuration view of a screw extruder B used in Examples 1 and 3, FIG. 1(C) is a configuration view of a screw extruder C used in Example 6, FIG. 1(D) is a configuration view of a screw extruder D used in Comparative Example 2, and FIG. 1(E) is a configuration view of a screw extruder E used in Comparative Examples 1 and 3.

FIG. 2 is a bird's eye view of the seal ring used in screw extruders A to E.

FIG. 3 is a side view of the seal ring used in screw extruders A to E.

### MODE FOR CARRYING OUT INVENTION

The present invention is described in detail below.

### [Type of Screw Extruder]

A screw extruder is used in the present invention. The screw extruder to be used may be any one of a single-screw extruder, a multi-screw extruder, and a special extruder. Of those, a multi-screw extruder, which can apply stronger shear to a biomass material, is preferred, and a twin-screw extruder is more preferred because of its generality and versatility.

As the multi-screw extruder, there may be adopted any one of a type in which screw shafts are parallel to one another and a conical type in which the screw shafts cross obliquely one another. Of those, a parallel type is preferred.

Any one of an engaged-screw type and a non-engaged-screw type may be adopted, but of those, an engaged-screw type is preferred because of a high kneading effect and many practical examples.

With regard to a screw rotation direction, any one of a co-rotation type and a counter-rotation type may be adopted, but of those, a co-rotation type is preferred because of a self-cleaning effect.

[Raw Material Feed Portion of Screw Extruder]

A hopper to be used for a raw material feed portion configured to stably feed a raw material to a cylinder of the screw extruder is not limited as long as the hopper has a function capable of generating a feed pressure required for a feed portion of a screw without causing bridging of raw materials, and examples of such hopper include a vibration hopper, a hopper with a force feeder, a hopper dryer, a vacuum hopper, and a nitrogen purge hopper. A hopper comprising a screw located on the inside of the hopper and configured to forcibly push a material into the cylinder is preferred from the viewpoint of stably feeding the raw material.

A device configured to quantitatively feed the raw material to the screw extruder is mounted below the hopper. The quantitative feeding device is not limited as long as the device has a function of enabling quantitative feeding, and examples of such device include a mass feeder and a constant volume feeder. Of those, a mass feeder is preferred in view of feeding raw material biomass, which generally has a low bulk density and non-uniform shapes and sizes. In order to feed the raw material to the screw extruder more securely, it is preferred to mount a compactor configured to forcibly press the raw material into the extruder through use of a screw or a piston so that the bulk density of the material can be increased.

[Cylinder Portion of Screw Extruder]

A cylinder portion of the screw extruder includes the following three sections: a heating section, which is located in the middle portion of the cylinder, and is configured to perform hydrothermal treatment through heating with a heater while grinding the raw material; a pulverization section, which is located upstream of the heating section, and is configured to pulverize the raw material and adjust its water content ratio, to thereby consolidate the material and maintain airtightness; and a cooling section, which is located downstream of the heating section, and is configured to cool the material, to thereby consolidate the material and maintain the airtightness. The screw extruder has an L/D of preferably from 30 to 80, more preferably from 40 to 80, still more preferably from 50 to 80 in its entirety including the pulverization section, the heating section, and the cooling section from the viewpoint of stably maintaining sealing, and performing hydrothermal treatment having an effect of improving the saccharification performance of the raw material biomass. The pulverization section has an L/D of preferably from 10 to 40, more preferably from 10 to 30, still more preferably from 15 to 25. In addition, the heating section has an L/D of preferably from 10 to 65, more preferably from 15 to 60, still more preferably from 20 to 55. The cooling section has an L/D of preferably from 5 to 35, more preferably from 5 to 20, still more preferably from 5 to 10. It should be noted that the “L/D” refers to an effective length represented by a ratio between the length (L) of a screw, which is measured from a start point of screw thread below the hopper to the tip of the screw, and the diameter (D) of the screw.

[Configuration of Pulverization Section of Cylinder]

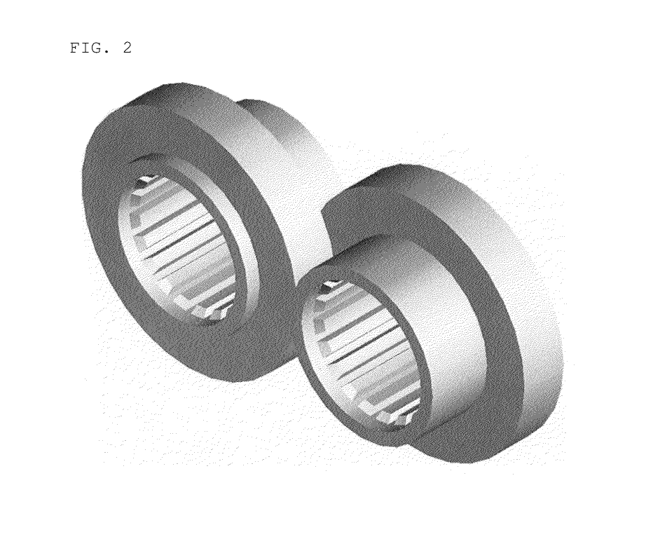

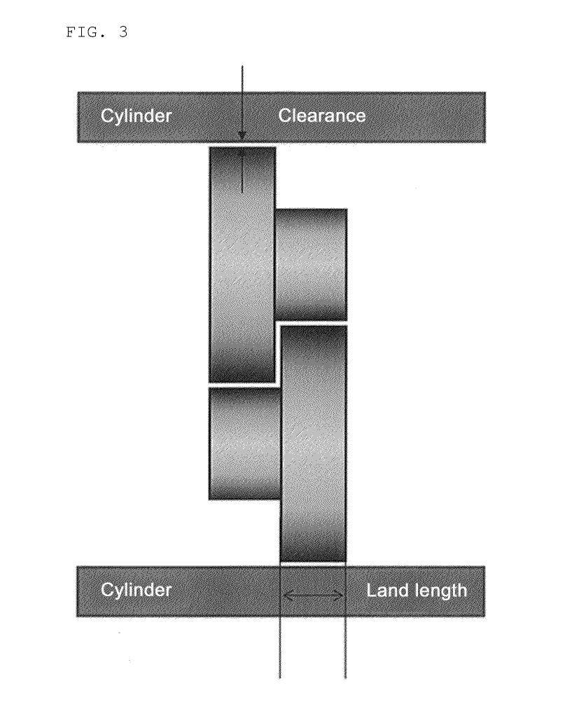

The pulverization section of the cylinder preferably has a screw configuration in which at least one or more elements each comprising a seal ring and at least one set of a kneading disc (feed kneading disc, neutral kneading disc, or reverse kneading disc) and/or a left-hand screw arranged upstream of the seal ring (hereinafter abbreviated as “seal ring elements”) are arranged. A state in which the raw material on an upstream side is compressed is achieved by a damming effect exhibited by the arranged seal ring, thereby achieving a state in which the shear force of a screw located upstream of the seal ring is increased. As a result, the raw material is efficiently pulverized and consolidated, and exhibits a function of sealing the pressure of vapor to be generated in the heating section. The seal ring has a shape as shown in the bird's eye view of FIG. 2, and is arranged in a cylinder in a twin-screw extruder as shown in the side view of FIG. 3. The pulverization of the raw material is performed not only for achieving the sealing function but also for improving efficiency of the hydrothermal treatment in the heating section. For this, the maximum grain size of the raw material is preferably set to 1,000 μm or less. It should be noted that the maximum grain size is determined through microscopic observation of a sample extracted from the pulverization section immediately upstream of the heating section.

In addition, in the pulverization section, the water content ratio of the raw material is adjusted to preferably from 30 mass % to 80 mass %, more preferably from 30 mass % to 75 mass %, still more preferably from 35 mass % to 70 mass % in order to optimally perform a hydrothermal reaction and achieve an optimal sealing property. The adjustment of the water content ratio may be separately performed before loading, but is preferably performed by installing a liquid feed line in an arbitrary portion of the pulverization section to feed water therethrough with a high-pressure pump from the viewpoint of reducing the number of steps. It should be noted that the water content ratio refers to the ratio of the mass of water to the total mass of the raw material as it is.

[Configuration of Heating Section of Cylinder]

In the present invention, it is critical to arrange two or more, preferably four or more sets of seal rings in the heating section. This enables efficient hydrothermal treatment of biomass to thereby obtain the above-mentioned effects of the invention.

The arrangement of a plurality of seal rings in the heating section exhibits such effect that strong grinding stress, which is generated when the cellulose-containing biomass serving as a raw material passes through an extremely narrow clearance between the seal ring and the cylinder, is applied concurrently with the hydrothermal treatment, thereby improving the saccharification performance of cellulose in biomass. For such effect, the clearance between the seal ring and the cylinder is preferably from 0.5% to 10.0%, more preferably from 1.0% to 8.0%, still more preferably from 1.5% to 5.0% with respect to the inner diameter of the cylinder. It should be noted that the inner diameter of the cylinder in a twin-screw extruder refers to the diameter of a circle surrounding one screw in a vertical cross section of the cylinder. The heating in the heating section is not limited as long as the cylinder can be heated, but is preferably performed with an electric heater from the viewpoint of temperature controllability. As the conditions of the hydrothermal treatment for the raw material, the temperature of the raw material falls within a range of preferably from 150° C. to 250° C., more preferably from 160° C. to 240° C., still more preferably from 170° C. to 230° C. A time of passage through the heating section falls within a range of preferably from 0.1 minute to 10 minutes, more preferably from 0.2 minute to 7.5 minutes, still more preferably from 0.3 minute to 7.5 minutes. A pressure in the heating section falls within a range of preferably from 0.1 MPa to 20 MPa, more preferably from 1 MPa to 15 MPa, still more preferably from 2 MPa to 12 MPa.

[Configuration of Cooling Section of Cylinder]

The cooling section of the cylinder preferably includes a water cooling jacket and/or a liquid feed line in order to cool the raw material heated in the heating section. The cooling in the cooling section is performed so that the temperature of the raw material is reduced to preferably 100° C. or less, more preferably 80° C. or less, still more preferably 70° C. or less. With this, vapor generated in the heating section turns into water, and the pressure of vapor flowing downstream together with the treated biomass can be sealed. Further, a pressure regulating valve may be mounted to a discharge port in the cooling section in order to seal the the pressure of vapor more stably in the system.

[Cellulose-Containing Biomass]

The biomass which can be treated with the screw extruder of the present invention means a biopolymer (nucleic acid, protein, or polysaccharide) or an industrial resource derived from such constituent component, other than exhaustible resources (fossil fuel, such as petroleum, coal, or natural gas). Therefore, examples of the cellulose-containing biomass include hard biomass such as wood, and soft biomass such as rice straw, wheat straw, corncobs, cassava, bagasse, or sugar cane leaves. Soft biomass is preferred in consideration of the ease of the pretreatment, and further, bagasse and sugar cane leaves are particularly preferred in consideration of their global storage potential and collection cost.

[Pulverization (Adjustment of Grain Size)]

The cellulose-containing biomass serving as a raw material may be directly fed to the screw extruder of the present invention without pulverization treatment, but is preferably subjected to adjustment of a grain size in advance through coarse pulverization before its feeding. Pulverization means is not particularly limited as long as the means has a function capable of pulverizing a solid substance. For example, the mode of the device may be a dry mode or a wet mode. In addition, the pulverization system of the device may be a batch system or a continuous system. Further, the pulverization force of the device may be provided by any of impact, compression, shearing, friction, and the like.

Specific examples of the device which can be used for the pulverization treatment include a coarse crusher, such as a shredder, a jaw crusher, a gyratory crusher, a cutter mill, a cone crusher, a hammer crusher, a roll crusher, or a roll mill; or a medium crusher, such as a stamp mill, an edge runner, a cutting/shearing mill, a rod mill, an autogenous mill, or a roller mill. Of those, a cutter mill is preferred from the viewpoints of a treatment amount and a pulverization range. The time for treating the raw material is not particularly limited as long as the raw material can be homogeneously and finely pulverized by the treatment.

The grain size of the raw material subjected to pulverization in advance before its feeding is preferably a size passing through a screen (sieve) having a screen diameter of from 0.5 mm to 30 mm because, when the discharge screen diameter of the pulverization device is excessively large, the grain size of the cellulose-containing biomass increases, resulting in high sugar production cost owing to a reduction in subsequent pretreatment effects, and when the screen diameter is excessively small, pulverization cost increases. The grain size is more preferably a size passing through a screen of from 1 mm to 30 mm, most preferably a size passing through a screen of from 3 mm to 30 mm. In addition, also in the case of performing pulverization without using a screen, it is preferred to pulverize the raw material so as to achieve a size corresponding to that of a pulverized product in the case of using the screen.

[Adjustment of Water Content Ratio]

The cellulose-containing biomass serving as a raw material may be subjected to adjustment of a water content ratio in advance before its feeding to the screw extruder of the present invention.

As a method of adjusting a water content ratio, there are given addition of water, dewatering, and drying, in accordance with the water content ratio of the raw material before the adjustment. As described above, the water content ratio of the raw material is preferably adjusted to from 30 mass % to 80 mass % in order to optimally perform the hydrothermal reaction and achieve an optimal sealing property.

An acid or alkali as an additive may be added to water in a raw material used in the treatment method with a screw extruder of the present invention, but it is industrially preferred to use only water, which is generally available, because the use of the additive not only increases chemical cost but also produces cost for detoxification, such as neutralization, in a subsequent step.

A biomass composition for saccharification can be efficiently produced by treating the biomass by the above-mentioned method. Further, when the biomass composition for saccharification produced by the above-mentioned method is hydrolyzed, a sugar can be efficiently produced.

### EXAMPLES

The present invention is hereinafter described by way of Examples and Comparative Examples. However, the present invention is by no means limited to the descriptions of Examples and Comparative Examples.

In Examples and Comparative Examples, screw extruders under five kinds of device conditions was used, and pretreatment of cellulose-containing biomass was conducted with changing the conditions of a hydrothermal temperature and a hydrothermal time. Further, a treated sample was evaluated for a saccharification rate for feeding the results back to select preferred device conditions of the screw extruder.

[Screw Extruder A]

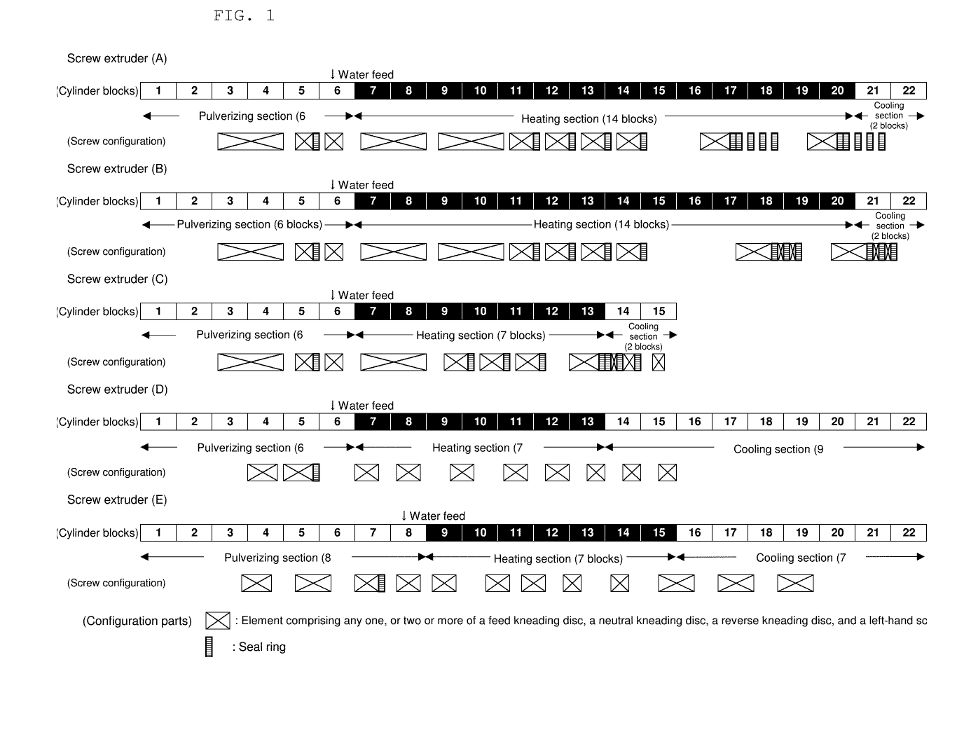

A screw extruder A in which a twin-screw extruder having an L/D of 77.0 and a screw diameter of 32 mm (trade name: TEX30α, manufactured by The Japan Steel Works, Ltd.) was allowed to have the following screw configuration illustrated in FIG. 1(A) and Table 1: a pulverization section in 6 blocks, a heating section in 14 blocks (6 sets of seal ring elements), and a cooling section in 2 blocks was used in Examples 2, 4 and 5.

[Screw Extruders B to E]

Screw extruders B to E (illustrated in FIG. 1(B) to FIG. 1(E), respectively) each having the same configuration as that of the screw extruder A except that the number of blocks of the pulverization section, the number of blocks of the heating section and the number of seal ring elements in the heating section, and the number of blocks of the cooling section were changed as shown in Table 1 were used in Examples and Comparative Examples shown in Table 1.

It should be noted that an L/D value, the number of blocks of sections other than the heating section, the number of seal ring elements in these sections, the number of seal rings used, and a clearance between a seal ring and a screw were also shown in Table 1. In addition, an extruder having a cylinder diameter of 47 mm (trade name: TEX44α, manufactured by The Japan Steel Works, Ltd.) was used as the screw extruder C.

TABLE 1

| | | | | | | | | | Number of seal rings | | | |
|---|---|---|---|---|---|---|---|---|---|---|---|---|
| | L/D | | | | Number of blocks | | | | used | | | |
| Extruder | Whole | Pulverization | Heating | Cooling | Pulverization | Heating | Cooling | | Pulverization | Heating | Cooling | |
| No. | device | section | section | section | section | section | section | Total | section | section | section | Total |
| Screw | 77 | 21 | 49 | 7 | 6 | 14 | 2 | 22 | 1 | 11 | 3 | 15 |
| extruder A | | | | | | | | | | | | |
| Screw | 77 | 21 | 49 | 7 | 6 | 14 | 2 | 22 | 1 | 7 | 3 | 11 |
| extruder B | | | | | | | | | | | | |
| Screw | 52.5 | 21 | 24.5 | 7 | 6 | 7 | 2 | 15 | 1 | 4 | 3 | 8 |
| extruder C | | | | | | | | | | | | |
| Screw | 77 | 21 | 24.5 | 31.5 | 6 | 7 | 9 | 22 | 1 | 0 | 0 | 1 |
| extruder D | | | | | | | | | | | | |
| Screw | 77 | 28 | 24.5 | 24.5 | 8 | 7 | 7 | 22 | 1 | 0 | 0 | 1 |
| extruder E | | | | | | | | | | | | |

| | | | | | Clearance | | | |
|---|---|---|---|---|---|---|---|---|
| | Number of seal ring | | | | between seal | | Diameter | Examples or |
| | elements | | | | ring and screw | | of | Comparative |
| Extruder | Pulverization | Heating | Cooling | | Length | | cylinder | Examples |
| No. | section | section | section | Total | (mm) | Ration (%) | (mm) | used |
| Screw | 1 | 6 | 0 | 7 | 0.5 | 1.6 | φ32 | Examples 2, |
| extruder A | | | | | | | | 4 and 5 |
| Screw | 1 | 5 | 1 | 7 | 0.5 | 1.6 | φ32 | Examples 1 |
| extruder B | | | | | | | | and 3 |
| Screw | 1 | 4 | 3 | 8 | 2.0 | 4.3 | φ47 | Example 6 |
| extruder C | | | | | | | | |
| Screw | 1 | 0 | 0 | 1 | 0.5 | 1.6 | φ32 | Comparative |
| extruder D | | | | | | | | Example 2 |
| Screw | 1 | 0 | 0 | 1 | 0.5 | 1.6 | φ32 | Comparative |
| extruder E | | | | | | | | Examples 1 |
| | | | | | | | | and 3 |

[Preparation of Raw Material Bagasse]

Bagasse was used as the cellulose-containing biomass serving as a raw material.

Bagasse subjected to no treatment (water content ratio: 50%, hereinafter abbreviated as “untreated bagasse”), and bagasse (water content ratio: 10.3%, hereinafter abbreviated as “3-mm bagasse”) prepared by pulverizing air-dried bagasse with a cutter mill having a screen diameter of 3 mm (MKCM-3, manufactured by Masuko Sangyo Co., Ltd.) were used as the bagasse.

[Analysis Method for Content Ratios of Main Components of Biomass]

The content ratio of cellulose, the content ratio of hemicellulose, and the total content ratio of lignin and an ash content in the biomass were determined by an analysis method (Technical Report NREL/TP-510-42618) of The National Renewable Energy Laboratory (NREL).

[High-performance Liquid Chromatography Analysis Method and Calculation Method for Content Ratio of Cellulose]

A guard column (KS-G manufactured by Showa Denko K. K.) and a separation column (KS-802 manufactured by Showa Denko K. K.) were connected to each other, and the column temperature was set to 75° C. Pure water was supplied as an eluting solution at a rate of 0.5 ml/min, and a separated component was subjected to quantitative determination with a differential refractive index detector. Thus, the concentration of glucose was determined, and the content ratio of cellulose was calculated based on the following equation.

Content ratio of cellulose (%)={mass of filtrate (g)×(concentration of glucose (%)/100)×0.9}/mass of weighed biomass (g)×100

The numerical value “0.9” in the equation is a coefficient for correcting changes in molecular weight caused by hydrolysis of cellulose.

[Measurement of Saccharification Performance with Enzyme]

Preparation of Acid Buffer Solution:

30 g of acetic acid was put in a 100 ml measuring flask, and diluted with pure water to give a 5 M acetic acid aqueous solution. 41 g of sodium acetate was put in a 100 ml measuring flask, and diluted with pure water to give a 5 M sodium acetate aqueous solution. The 5 M acetic acid aqueous solution was added to the 5 M sodium acetate aqueous solution until the pH became 5.0. Thus, an acetic acid buffer solution was obtained.

Preparation of Enzyme Solution:

1.5 g of Meicelase (trademark, cellulase manufactured by Meiji Seika Kaisha, Ltd. (currently Meiji Seika Pharma Co., Ltd.)) was dissolved in 98.5 g of pure water.

The FPU activity (Filter Paper Assay for Saccharifying Cellulase) of the enzyme solution was 6 FPU/g, which was determined according to an analysis method of International Union of Pure and Applied Chemistry (IUPAC) (Pure & Appl. Chem., Vol. 59, No. 2, pp. 257-268, 1987).

Saccharification Reaction:

A rotor was put in a 50 ml glass vessel with a cover, and a composition subjected to pretreatment was weighed so that the amount of cellulose was 0.5 g. Then, 0.6 g of the acetic acid buffer solution and 1.03 g of the enzyme solution were added thereto, and further, pure water was added thereto to give a total of 10 g. The resultant was subjected to a saccharification reaction with the enzyme in a thermostat bath at 40° C. for 72 hours (Hr) while being stirred. The resultant saccharified solution was subjected to quantitative determination for glucose by high-performance liquid chromatography analysis. Thus, a saccharification rate and a sugar utilization rate were calculated by the following equations.

Saccharification rate (%)={concentration of glucose (%) in reaction solution×0.9}/concentration of cellulose (%) in reaction solution at the beginning of reaction}×100

The numerical value “0.9” in the equation is a coefficient for correcting changes in molecular weight caused by hydrolysis of cellulose.

### Example 1

The 3-mm bagasse was loaded into the screw extruder B having a screw rotation number of 350 rpm with a mass feeder and a compactor at a feed rate of 5.0 kg/Hr of a mass of the bagasse as it was and 4.5 kg/Hr in terms of dry mass. In the pulverization section, water was added through a liquid feed line at a feed rate of 4.8 kg/Hr, and a water content ratio was adjusted so that a water content ratio of 54 mass % was continuously obtained in the pulverization section upstream of the heating section. The raw material was subjected to hydrothermal treatment in the heating section so that the temperature of the raw material in the heating section and a pressure in the heating section were set to 175° C. and 5 MPa, respectively, and then cooled to 70° C. or less in the cooling section with a water cooling jacket, followed by recovery of a sample from a discharge port. A time of passage through the heating section under such conditions was 7.5 minutes. 138 g of water, which was three times the amount of a solid content (46 mass %) in the sample, was added to 100 g of the treated sample recovered, and the resultant was suspended, followed by centrifugal filtration with a centrifugal filter (H-122, manufactured by Kokusan Co., Ltd., filter cloth: cotton) at 3,000 rpm. Thus, a water-containing solid content was obtained. The obtained water-containing solid content was calculated for a saccharification rate by the above-mentioned methods.

### Examples 2 to 6 and Comparative Examples 1 to 3

The saccharification rate was evaluated in the same manner as in Example 1 except that the raw materials, device conditions and treatment conditions were changed as shown in Table 2. The results are shown in Table 2.

TABLE 2

| | Raw material | | | | | | | | |
|---|---|---|---|---|---|---|---|---|---|
| | | Solid | Device conditions | | | | Treatment conditions | | |
| | | content | | | | Number of | Feed rate | | Water |
| | | concentration | | | Number of | seal ring | | Mass in | feed rate |
| | | of | Screw | | seal rings | elements | | terms of | in the |
| | | the raw | rotation | | used in the | in the | Mass | dry | liquid |
| | Kind of | material | number | Screw | heating | heating | as it is | mass | feed line |
| | bagasse | [mass %] | [rpm] | extruder | section | section | [kg/Hr] | [kg/Hr] | [kg/Hr] |
| Example 1 | 3-mm | 90 | 350 | B | 7 | 5 | 5.0 | 4.5 | 4.8 |
| | bagasse | | | | | | | | |
| Example 2 | 3-mm | 90 | 350 | A | 11 | 6 | 5.0 | 4.5 | 3.6 |
| | bagasse | | | | | | | | |
| Example 3 | 3-mm | 90 | 350 | B | 7 | 5 | 5.0 | 4.5 | 4.8 |
| | bagasse | | | | | | | | |
| Example 4 | 3-mm | 90 | 350 | A | 11 | 6 | 5.0 | 4.5 | 2.8 |
| | bagasse | | | | | | | | |
| Example 5 | 3-mm | 90 | 350 | A | 11 | 6 | 12.5 | 11.3 | 5.8 |
| | bagasse | | | | | | | | |
| Example 6 | Untreated | 50 | 200 | C | 4 | 4 | 16.5 | 8.3 | 4.1 |
| | bagasse | | | | | | | | |
| Comparative | 3-mm | 90 | 350 | E | 0 | 0 | 2.5 | 2.3 | 2.9 |
| Example 1 | bagasse | | | | | | | | |
| Comparative | 3-mm | 90 | 350 | D | 0 | 0 | 2.5 | 2.3 | 2.9 |
| Example 2 | bagasse | | | | | | | | |
| Comparative | 3-mm | 90 | 350 | E | 0 | 0 | 2.5 | 2.3 | 2.0 |
| Example 3 | bagasse | | | | | | | | |

| | Treatment conditions | | | | | |
|---|---|---|---|---|---|---|
| | Water content | | | | Water in an | |
| | in the | | | | amount of | Treatment |
| | pulverizing | | | | three times the | result |
| | section (solid | Hydro- | | Hydro- | amount of a | Sugar |
| | content | thermal | | thermal | solid content | utilization |
| | concentration) | temperature | Pressure | time | of the sample | rate |
| | [mass %] | [° C.] | [MPa] | [min.] | [g] | [%] |
| Example 1 | 54 (46) | 175 | 5 | 7.5 | 138 | 66 |
| Example 2 | 48 (52) | 190 | 5 | 7.5 | 156 | 74 |
| Example 3 | 54 (46) | 190 | 5 | 7.5 | 138 | 70 |
| Example 4 | 42 (58) | 215 | 5 | 7.5 | 174 | 62 |
| Example 5 | 42 (58) | 220 | 5 | 3.0 | 186 | 67 |
| Example 6 | 66 (34) | 220 | 5 | 3.0 | 100 | 52 |
| Comparative | 58 (42) | 175 | 5 | 7.5 | 126 | 29 |
| Example 1 | | | | | | |
| Comparative | 58 (42) | 190 | 5 | 7.5 | 126 | 34 |
| Example 2 | | | | | | |
| Comparative | 50 (50) | 215 | 5 | 7.5 | 150 | 37 |
| Example 3 | | | | | | |

[Operation Results in Terms of Conditions of Cooling Section]

Examples and Comparative Examples in this description were test examples in which the hydrothermal treatment was able to be performed with a screw extruder. In each screw extruder, a cooling system of a water cooling jacket and a liquid feed line was used in the cooling section, and a pressure regulating valve was mounted to a discharge port. In the test examples, the temperature in the cooling section was reduced to 70° C. or less through cooling with a water cooling jacket in Examples 1 to 5 and Comparative Examples 1 to 3 and through cooling with the water cooling jacket and water feeding from a liquid feed line to the biomass to be treated in combination in Example 6. The hydrothermal treatment was able to be stably and continuously performed in all the test examples.

[Comparison of Saccharification Performance in Terms of Treatment Conditions]

The saccharification rate under the same hydrothermal conditions of the heating temperature at 175° C. and retention time in 7.5 minutes resulted in 66% in Example 1 and 29% in Comparative Example 1, and the saccharification rate under the same hydrothermal conditions of the heating temperature at 190° C. and retention time in 7.5 minutes resulted in 74% in Example 2 and 34% in Comparative Example 2. And further, the saccharification rate under the same hydrothermal conditions of the heating temperature at 215° C. and retention time in 7.5 minutes resulted in 62% in Example 4 and 37% in Comparative Example 3.

The treatment in each of these examples was conducted with an extruder comprising a screw for kneading in the heating section. However, when the number of seal ring elements is examined, while 5 sets in Examples 1 and 3, 6 sets in Examples 2, 4 and 5, and 4 sets in Example 6 are arranged, no element is arranged in any of Comparative Examples 1 to 3. Accordingly, it was confirmed that the extreme reduction in the saccharification rate under the same hydrothermal conditions is caused by the absence of the seal ring element.

A difference in the saccharification rate depending on the number of seal ring elements to be included was considered under the same hydrothermal conditions. As a result, it was found that, under the hydrothermal conditions of the heating temperature at 190° C. and the retention time in 7.5 minutes, the saccharification rate was 74% in Example 2 including 6 sets of seal ring elements, whereas the saccharification rate was 70% in Example 3 including 5 sets of seal ring elements, and under the hydrothermal conditions of the heating temperature at 220° C. and retention time in 3.0 minutes, the saccharification rate was 67% in Example 5 including 6 sets of seal ring elements, whereas the saccharification rate was 52% in Example 6 including 4 sets of seal ring elements. It was suggested that the saccharification rate depended on the number of the seal ring element. Such effect exhibited by the seal ring element is presumed to be caused as follows: high grinding stress is applied to the cellulose-containing biomass serving as a raw material when it passes through an extremely narrow clearance portion between the seal ring and the cylinder concurrently with the hydrothermal treatment, and thus the saccharification performance of cellulose in the biomass is improved.

From the above-mentioned results, it was confirmed that a cellulose-containing composition exhibiting a high saccharification rate can be obtained by a screw extrude in which a plurality of seal ring elements in the heating section.

While seal rings are arranged in screw extruders A to C, no seal ring is arranged in screw extruders D and E. From the fact that the treatment is conducted at a constant pressure in Examples and Comparative Examples, and seal rings are arranged in the pulverization section of all the screw extruders, it can be confirmed that the arrangement of seal rings in the heating section of the screw extruder is a direct cause of improvement in the saccharification rate.

1. A screw extruder, which is a screw extruder for continuously conducting hydrothermal treatment of cellulose-containing biomass to produce a biomass composition for saccharification, and comprises a raw-material feeding section, a pulverization section, a heating section and a cooling section, characterized in that a plurality of seal rings are provided in the heating section.

2. The screw extruder as claimed in claim 1, wherein three sets or more elements each comprising a seal ring and at least one set of a kneading disc and/or a left-hand screw arranged immediately upstream of the seal ring are arranged in the heating section.

3. The screw extruder as claimed in claim 2, wherein the clearance between the seal ring arranged in the heat section and a cylinder is 0.5% to 10.0% with respect to the inner diameter of the cylinder.

4. The screw extruder as claimed in claim 1, wherein at least one or more elements each comprising a seal ring and at least one set of a kneading disc and/or a left-hand screw is arranged in the pulverization section.

5. The screw extruder as claimed in claim 1, wherein a liquid feed line is arranged in the pulverization section.

6. The screw extruder as claimed in claim 1, wherein a water cooling jacket and/or a liquid feed line are arranged in the cooling section.

7. The screw extruder as claimed in claim 1, wherein a compactor is provided in the raw-material feeding section.

8. The screw extruder as claimed in claim 1, wherein an L/D as being an effective length represented by a ratio between the length (L) of a screw, which is measured from a start point of screw thread below the hopper to the tip of the screw, and the diameter (D) of the screw, is from 30 to 80 in its entirety including the pulverization section, the heating section and cooling section, and from 10 to 40 in the pulverization section.

9. The screw extruder as claimed in claim 1, wherein the screw extruder is a co-rotating twin-screw extruder.

10. The screw extruder as claimed in claim 1, wherein the cellulose-containing biomass is soft biomass.
